(19) United States
(12) Patent Application Publication
(10) Pub. No.: US 2016/0318826 A1
(43) Pub. Date: Nov. 3, 2016

(54) TUBE SHAPED CARBON STRUCTURE AND MANUFACTURING METHOD THEREOF

(71) Applicants: INSTITUTE FOR BASIC SCIENCE, Daejeon (KR); POSTECH ACADEMY-INDUSTRY FOUNDATION, Pohang-si (KR)

(72) Inventors: Hee Cheul CHOI, Pohang-si (KR); Jungah KIM, Pohang-si (KR); Chibeom PARK, Pohang-si (KR)

(21) Appl. No.: 14/872,921

(22) Filed: Oct. 1, 2015

(30) Foreign Application Priority Data
Apr. 30, 2015 (KR) 10-2015-0061495

(51) Int. Cl. C07C 2/86; C07C 13/62

(52) U.S. Cl. CPC C07C 2/86; C07C 13/62; C07C 2104/00

(57) ABSTRACT

The present invention relates to a tube shaped carbon structure and a manufacturing method thereof.

### CROSS-REFERENCE TO RELATED APPLICATION

This application claims priority to Korean Patent Application No. 10-2015-0061495 filed on Apr. 30, 2015, and all the benefits accruing therefrom under 35 U.S.C. §119, the contents of which is incorporated by reference in its entirety.

### BACKGROUND OF THE INVENTION

1. Field of the Invention

The present invention relates to a tube shaped carbon structure and a manufacturing method thereof.

2. Description of the Related Art

Manufacturing a light source emitting light from an organic molecule or an organic molecule system is an important prerequisite for mass producing next generation electronic devices and photoelectronic devices at low costs. In addition, when the organic light source is used, the component of the next generation electronic device and the photoelectronic device is expected to be made light-weight and flexible.

The light emission occurring from an exciton recombination requires a specific energy bandgap, and for the organic light source to satisfy this condition, the organic molecule having a high level of conjugated double bond needs to exist in a crystalline structure.

Fullerene including C₆₀ (Buckmisterfullerene) and C₇₀ is a carbon allotrope, in which the entire molecule is formed in a conjugated double bond, and has a specific energy bandgap, and the bandgap may be easily controlled by doping, so fullerene including C₆₀ (Buckmisterfullerene) and C₇₀ is receiving much attention. In addition, the fullerene may have semiconductivities and superconductivities, so the fullerene is expected to be a promising electronic device material of the future. Meanwhile, in spite of the excellent electrical characteristics, the fullerene has a poor photoluminescence characteristic, so the fullerene has limits for use as a light source.

Recently, research results indicating that the fluorescence characteristics are improved when C₆₀ exists in a specific ensemble state in a powder form or a liquid form have become known and received attention. In addition, the C₇₀ has an elliptical molecular shape, and similar energy states coexist at a room temperature, thus controlling the crystallization of C₇₀ is difficult, so, compared to C_{H}, research for C₇₀ structure having a controlled shape is lacking.

As a related art, there is Korean Unexamined Patent Publication No. 10-2009-0120994 (Published on Nov. 25, 2009), ‘Fullerene-silica nano particle having improved fluorescent characteristic, a method of fabricating the same and a method of using the same’.

### SUMMARY OF THE INVENTION

Therefore, the present invention provides a tube shaped carbon structure having improved light emitting characteristics by using C₇₀ carbon powder and a manufacturing method thereof.

Objects of the present invention may not be limited to the above objects, and other objects will be clearly understandable to those having ordinary skill in the art from the disclosures provided below.

To solve the above mentioned problems, the present invention provides a tube shaped carbon structure including: C₇₀ and mesitylene, wherein C₇₀ molecules have a hexagonal structure, the mesitylene is bonded between the c₇₀ molecules and the hexagonal structures are regularly combined to form a tube shape.

In addition, the present invention provides a method of manufacturing a tube shaped carbon structure, the method including: adding a carbon powder into a mesitylene solution and irradiating an ultrasonic wave to dissolve the carbon powder; and adding an isopropyl alcohol solution into the mesitylene solution, in which the carbon powder is dissolved, by a volume ratio of 1:15 to 1:64, irradiating the ultrasonic wave and maintaining a stationary state.

According to the present invention, C₇₀ carbons are grown in a direction of [001] so that the C₇₀ carbon powder has high regularity and has a hexagonal single crystalline structure and a tube form, thereby having an improved light emitting characteristics compared to the C₇₀ carbon powder.

In addition, by controlling the volume ratio of the mesitylene solution, in which carbon powders are dissolved, and the isopropyl alcohol solution to 1:15 to 1:64, tube shaped carbon structure, in which a mesitylene molecule is included between carbon lattice, can be manufactured and the long-range order of the carbon molecules can be maintained by the high crystallizability.

### BRIEF DESCRIPTION OF THE DRAWINGS

FIG. 1 is a flowchart showing a method of manufacturing a tube shaped carbon structure according to the present invention.

FIG. 2 is a schematic diagram showing a growth mechanism of the tube shaped carbon structure in the method of manufacturing a tube shaped carbon structure according to the present invention.

FIG. 3a is a photographic view photographed by a scanning electron microscope and showing a carbon structure manufactured in comparative example 4.

FIG. 3b is a photographic view photographed by a scanning electron microscope and showing a carbon structure manufactured in embodiment 2 according to the present invention.

FIG. 3c is a photographic view photographed by a transmission electron microscope and showing the carbon structure manufactured in embodiment 2 according to the present invention.

FIG. 4a is a view showing an X-ray diffraction result of the C₇₀ structure manufactured in comparative example 4 and embodiment 2 according to the present invention.

FIG. 4b is a view showing a selected area electronic diffraction result of the C₇₀ structure manufactured in embodiment 2 according to the present invention and a bright field TEM picture showing the C₇₀ structure manufactured in embodiment 2 according to the present invention.

FIG. 4c is a graph showing a light emission spectrum of the C₇₀ structure manufactured in embodiment 2 according to the present invention.

FIG. 4d is a photographic view showing a light emission of the C₇₀ structure manufactured in embodiment 2 according to the present invention.

FIG. 4e is a photographic view showing a light emission of the C₇₀ powder.

FIG. 5 is a photographic view showing a shape of a carbon structure according to a volume ratio of a mesitylene solution, in which C₇₀ is dissolved, and an isopropyl alcohol solution of the carbon structure manufactured by the method of manufacturing a carbon structure according to the present invention.

### DETAILED DESCRIPTION OF THE INVENTION

Hereinafter, exemplary embodiment according to the present invention is described in detail with reference to the accompanying drawings.

Advantages and features of the present invention, and method for achieving thereof will be apparent with reference to the examples that follow.

But, it should be understood that the present invention is not limited to the following embodiments and may be embodied indifferent ways, and that the embodiments are given to provide complete disclosure of the invention and to provide thorough understanding of the invention to those skilled in the art, and the scope of the invention is limited only by the accompanying claims and equivalents thereof.

In addition, when describing embodiments of the present invention, detailed descriptions of well-known functions and structures incorporated herein may be omitted when they make the subject matter of the present invention unclear.

The present invention provides a tube shaped carbon structure including C₇₀ and mesitylene, wherein C₇₀ molecules have a hexagonal structure, the mesitylene is bonded between the C₇₀ molecules and the hexagonal structures are regularly combined to form a tube shape.

In the carbon structure according to the present invention, C₇₀ carbons are grown in a direction of [001] such that the carbon structure has high regularity and has a hexagonal single crystalline structure and a tube form, thereby having improved light emitting characteristics compared to the C₇₀ carbon powder.

In the carbon structure according to the present invention, the mole ratio of the C₇₀ to the mesitylene is 1:0.7, and the tube shaped carbon structure has an average diameter of 0.2 μm to 3.0 μm and an average length of 2 μm to 80 μm.

In addition, the present invention provides a method of manufacturing a tube shaped carbon structure, the method including: adding a carbon powder into a mesitylene solution, irradiating an ultrasonic wave to dissolve the carbon powder; and adding an isopropyl alcohol solution into the mesitylene solution, in which the carbon powder is dissolved, by a volume ratio of 1:15 to 1:64, irradiating the ultrasonic wave and maintaining the solution in a stationary state.

In the method of manufacturing a tube shaped carbon structure according to the present invention, by controlling the volume ratio of the mesitylene solution, in which the carbon powder is dissolved, to the isopropyl alcohol solution to 1:15 to 1:64, the tube shaped carbon structure, in which a mesitylene molecule is included between a carbon lattice structure, may be manufactured. In addition, the tube shaped carbon structure manufactured by the manufacturing method according to the present invention has an improved light emission characteristic because the long-range order of the carbon molecules is maintained due to the high crystallizability.

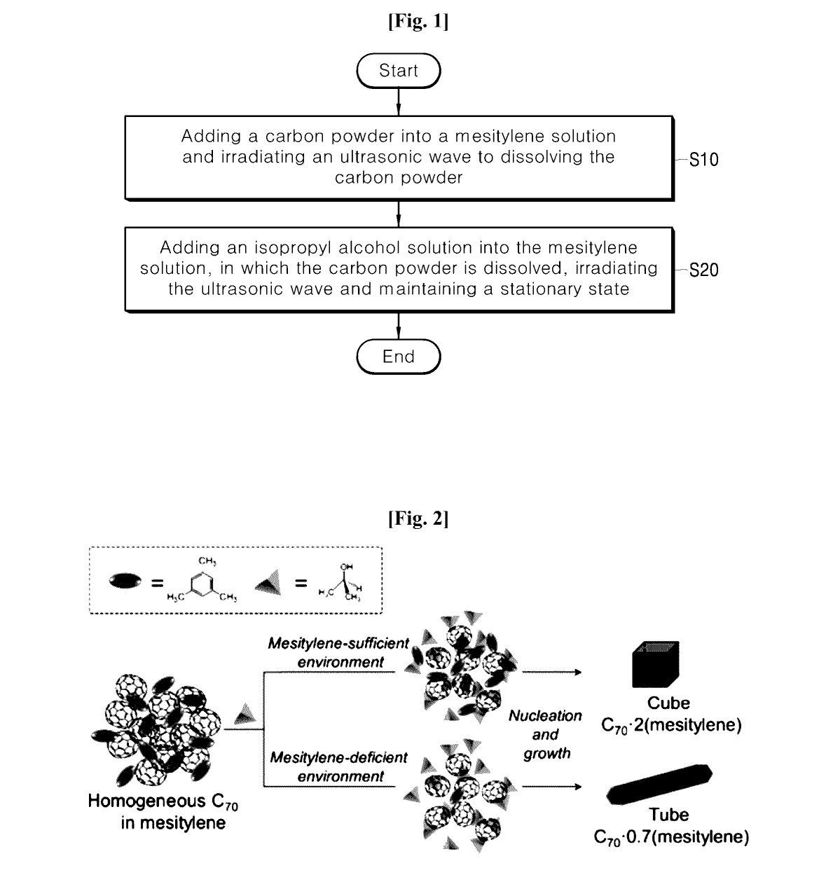

FIG. 1 is a flow chart showing a method of manufacturing a tube shaped carbon structure according to the present invention. Hereinafter, the present invention is described in detail with reference to FIG. 1.

The method of manufacturing a tube shaped carbon structure according to the present invention includes: adding a carbon powder into a mesitylene solution and irradiating an ultrasonic wave to dissolve the carbon powder (S10).

In the method of manufacturing a tube shaped carbon structure according to the present invention, the carbon is C₇₀, and the carbon concentration in the mesitylene solution is preferably 0.1 to 0.3 mg/mL. When the carbon concentration is less than 0.1 mg/mL, the solution does not achieve a supersaturated state sufficient enough for a crystal to be formed, so the carbon structure is not manufactured, and when the carbon concentration exceeds 0.3 mg/mL, the crystal is formed in a rod shape instead of a tube shape.

In addition, the carbon powder may be quickly dissolved in the mesitylene solution through the ultrasonic wave irradiation.

Next, the method of manufacturing a tube shaped carbon structure according to the present invention includes: adding an isopropyl alcohol solution into the mesitylene solution, irradiating the ultrasonic wave and maintaining the solution in a stationary state (S20).

In the method of manufacturing a tube shaped carbon structure according to the present invention, a cube or a cube shape may be manufactured according to the volume ratio of the mesitylene solution, in which the carbon powder is dissolved, to the isopropyl alcohol solution.

In more detail, in the method of manufacturing a tube shaped carbon structure according to the present invention, by adding the isopropyl alcohol solution by a volume ratio of 1:1 to 1:7 with respect to the mesitylene, a cube shaped carbon structure may be manufactured, and by adding the isopropyl alcohol solution by a volume ratio of 1:15 to 1:64 with respect to the mesitylene solution, a tube shaped carbon structure, in which both ends are empty, may be manufactured. In this case, when the volume ratio is 1:9 to 1:13, both cube shape and tube shape are created, so the volume ratio of 1:9 to 1:13 is a boundary value for manufacturing the cube shape or the tube shape.

The mesitylene solution and the isopropyl alcohol solution have excellent miscibility with each other, so they may be quickly mixed even by irradiating ultrasonic wave for only few seconds.

When the solution is maintained in a stationary state, the crystal precipitates, and the solution left on the precipitated crystals may be evaporated by using nitrogen gas.

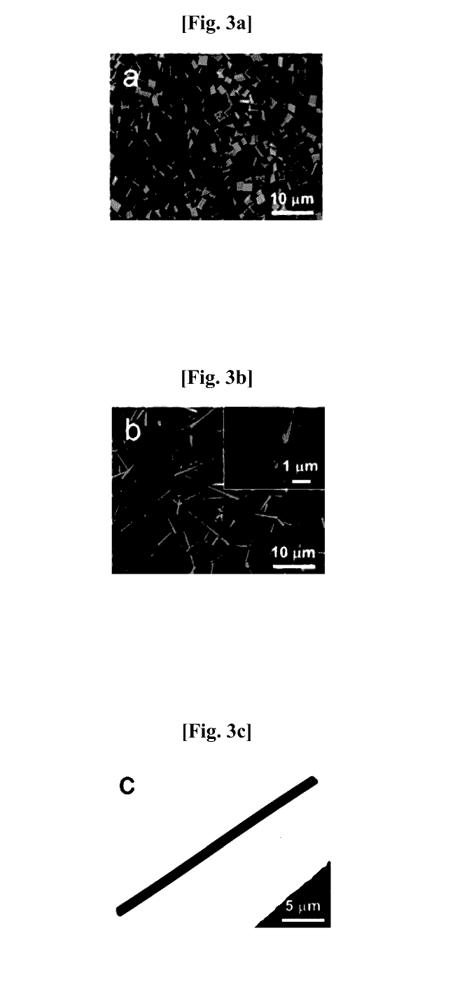

FIG. 2 is a schematic diagram showing a growth mechanism of the tube shaped carbon structure in the method of manufacturing a tube shaped carbon structure according to the present invention. Referring to FIG. 2, when the mesitylene solution and the isopropyl alcohol solution are uniformly mixed, the carbon concentration passes the saturation point, thereby creating nucleus in an amount controlled by the concentration of the carbon dissolved in the mesitylene solution. Then, during the crystallization process, the mesitylene molecule near the carbon molecule is inserted as a second component through a chemical interaction such as van der Waals and p-p interactions. In this process, the absolute amount of the mesitylene existing near the carbon plays an important role in the crystallization, and is effectively controlled by the relative amount of the isopropyl alcohol. Therefore, in an environment of sufficient mesitylene, the carbon and the mesitylene are combined by a mole ratio of 1:2 and the carbon molecules are crystallized such that the cube shaped carbon structure is manufactured, and in an environment of insufficient mesitylene, the carbon and the mesitylene are combined by a mole ration of 1:0.7 and the carbon molecules are crystallized such that the tube shaped carbon structure is manufactured.

### Embodiments 1 to 4

### Manufacturing the Tube Shaped C₇₀ Structure

C₇₀ powder (purchased from MTR, purity of 99.0%) was added into mesitylene and ultrasonic wave was irradiated for 3 hours to manufacture a mesitylene solution in which C₇₀ was dissolved. In this case, the concentration of the C₇₀ was 0.1 mM. Then, isopropyl alcohol (isopropanol) solution was added into the mesitylene solution, in which C₇₀ was dissolved, ultrasonic wave was irradiated for 30 seconds, and then the solution was maintained in a stationary state for 12 hours. The solution left on the precipitated crystals was evaporated by using nitrogen gas such that a tube shaped C₇₀ structure was manufactured. In this case, the mesitylene solution, in which C₇₀ was dissolved, was mixed with the isopropyl alcohol solution by a volume ratio of 1:15 to 1:64.

### Comparative Examples 1 to 5

### Manufacturing a Cube Shaped C₇₀ Structure

Other than mixing the mesitylene solution, in which C₇₀ was dissolved, with the isopropyl alcohol solution by a volume ratio of 1:1 to 1:7, the comparative examples were performed identical to the above embodiments.

Table 1 shows the volumes of the mesitylene solution, in which C₇₀ was dissolved, and the isopropyl alcohol solution, the volume ratio between the mesitylene solution, in which C₇₀ was dissolved, and the isopropyl alcohol solution, and the concentration of C₇₀.

TABLE 1

| | Volume of | Volume of | |
|---|---|---|---|
| | mesitylene solution | isopropyl | |
| | in which C₇₀ | alcohol | |
| | is dissolved | solution | Volume |
| Embodiments | (mL) | (mL) | ratio |
| Embodiment 1 | 10 | 10 | 1:1 |
| Embodiment 2 | 7.5 | 12.5 | 1:1.7 |
| Embodiment 3 | 5 | 15 | 1:3 |
| Embodiment 4 | 4 | 16 | 1:4 |
| Embodiment 5 | 2.5 | 17.5 | 1:7 |
| Embodiment 6 | 1.2 | 18.8 | 1:15 |
| Embodiment 7 | 0.65 | 19.35 | 1:30 |
| Embodiment 8 | 0.6 | 19.4 | 1:31 |
| Embodiment 9 | 0.3 | 19.7 | 1:64 |

### Experimental Example 1

### Structural Analysis of C₇₀ Structure

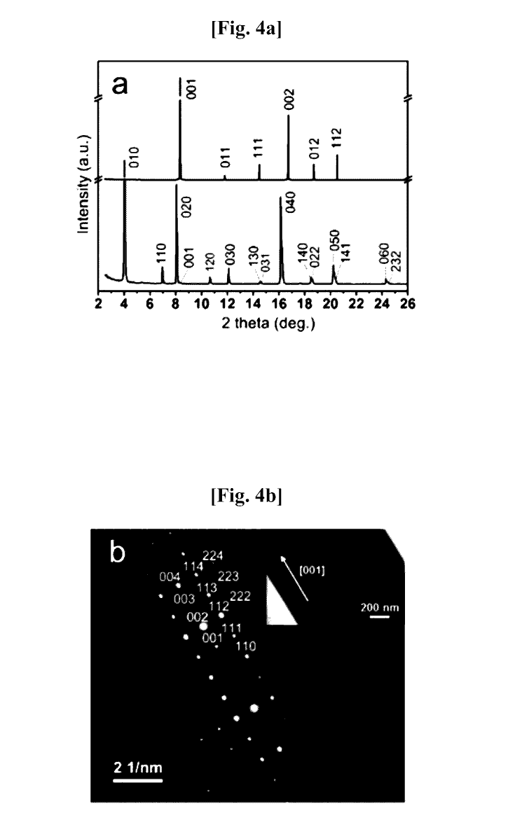

To identify the shape of the C₇₀ structure manufactured by the method of manufacturing a carbon structure according to the present invention, a scanning electron microscope (SEM) and a transmission electron microscope (TEM) were used and the results are shown in FIG. 3.

FIG. 3a is a photographic view photographed by a scanning electron microscope showing a carbon structure manufactured in comparative example 4. As shown in FIG. 3a, when the mesitylene solution, in which C₇₀ was dissolved, and the isopropyl alcohol solution were mixed by a volume ratio of 1:4, the cube shaped C₇₀ crystal was manufactured and the average crystal size was 3 μm.

In addition, FIG. 3b is a photographic view photographed by a scanning electron microscope showing a carbon structure manufactured in embodiment 2 according to the present invention, and the mesitylene solution, in which C₇₀ was dissolved, and the isopropyl alcohol solution were mixed by a volume ratio of 1:30 to manufacture a tube shaped C₇₀ crystal. As shown in FIG. 3 (b), the average diameter of the C₇₀ crystal was 800 nm, and the average length was 20 μm.

FIG. 3c is a photographic view photographed by a transmission electron microscope showing the carbon structure manufactured in embodiment 2 according to the present invention. As shown in FIG. 3c, the center part of the tube shaped C₇₀ crystal is filled, but the inner spaces of both ends of the crystal are empty. This shape may be formed because the edge parts of the tube grow more quickly than the inside of the tube, and the tube in which the center part is blocked may be formed because the concentration of C₇₀ at a part close to the central part of a seed is insufficient.

### Experimental Example 2

### Crystal Structure Analysis of C₇₀ Structure

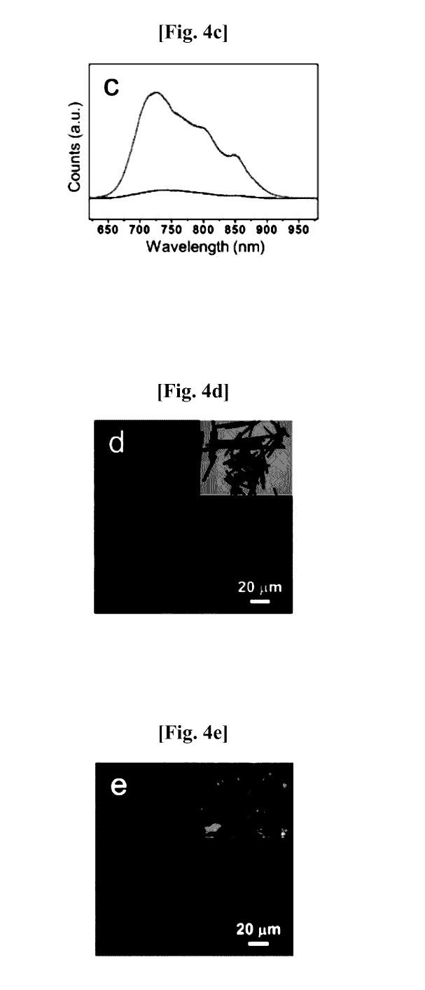

To identify the crystal structure of the C₇₀ structure manufactured by the method of manufacturing the carbon structure according to the present invention, an X-ray diffraction analysis was performed and the results are shown in FIG. 4.

FIG. 4a is a view showing an X-ray diffraction result of the C₇₀ structure manufactured in comparative example 4 and embodiment 2 according to the present invention. As shown in FIG. 4a, strong diffraction peaks occur in both of the cube shaped C₇₀ structure (upper XRD result) and the tube shaped C₇₀ structure (lower XRD result), however, the XRD patterns are entirely different, and the tube shaped C₇₀ structure is different from any other C₇₀ structures. When the C₇₀ structure was analyzed by a plane-spacing equation using Bragg rule, the tube shaped C₇₀ structure had a hexagonal shape having a lattice constant of a=25.36 Å, c=10.58 Å (a/c=2.40).

FIG. 4b is a view showing a selected area electronic diffraction result of the C₇₀ structure manufactured in embodiment 2 according to the present invention and a bright field TEM picture showing the C₇₀ structure manufactured in embodiment 2 according to the present invention. As shown in FIG. 4b, the tube shaped C₇₀ structure is a single crystal and has a growth axis of [001].

FIG. 4c is a graph showing a light emission spectrum of the C structure manufactured in embodiment 2 according to the present invention, the black line is a light emission spectrum of the C₇₀ powder, and the red line is the light emission spectrum of the C₇₀ structure manufactured in embodiment 2. As shown in FIG. 4c, the C₇₀ structure has an improved light emission characteristic due to the high crystallizability, and the light emission peak position is not largely different from the cube shaped C₇₀ structure.

FIG. 4d is a photographic view showing a light emission of the C₇₀ structure manufactured in embodiment 2 according to the present invention, and FIG. 4e is a photographic view showing a light emission of the C₇₀ powder. As shown in FIGS. 4d and 4e, the C₇₀ structure manufactured in embodiment 7 according to the present invention has a more excellent light emission characteristic than the C₇₀ powder.

### Experimental Example 3

### Analysis of the Shape of the Carbon Structure According to the Volume Ratio of the Solution

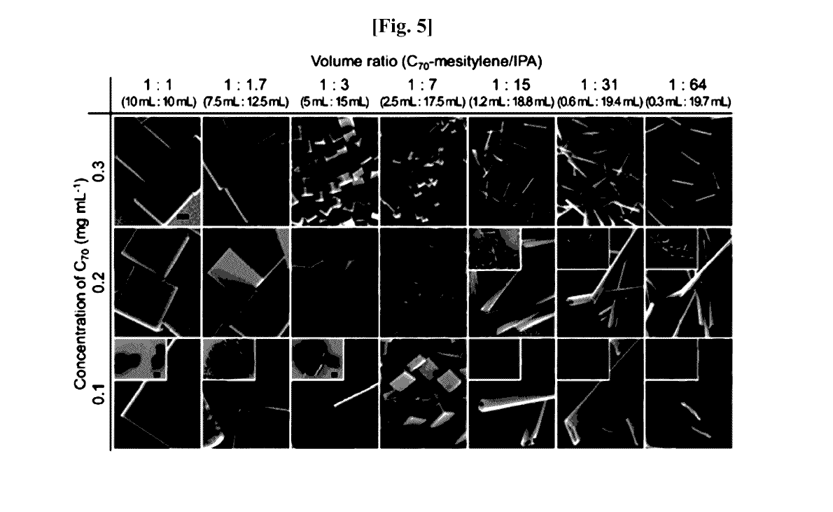

The shape of a carbon structure according to a volume ratio of a mesitylene solution, in which C₇₀ is dissolved, and an isopropyl alcohol solution of the carbon structure manufactured by the method of manufacturing a carbon structure according to the present invention is analyzed, and the results are shown in FIG. 5.

In this case, the volume of the entire solution was 20 mL, and the concentration of the C₇₀ was 0.1 mg/mL to 0.3 mg/mL.

As shown in FIG. 5, the form of the C₇₀ structure is maintained until the volume of the isopropyl alcohol solution increases to 1 to 7, and when the volume increases to 15 or higher, the amount of the mesitylene solution is reduced and tube shaped crystals are formed. In addition, similar tendencies occur when the concentration of the C₇₀ is 0.2 mg/mL or 0.1 mg/mL. However, the most distinctive difference is that when the concentration of the C₇₀ increases, the size of the cube of the diameter of the cube increases, and when the concentration of the C₇₀ decreases, the nucleus forming position decreases.

While the tube shaped carbon structure and a manufacturing method thereof of the present invention has been particularly shown and described with reference to various embodiments thereof, it will be understood by those of ordinary skill in the art that various substitutions, changes in form and alterations may be made therein without departing from the spirit and the scope of the present invention

Therefore, the scope of the present invention is not limited to the described embodiments, but is limited only by the accompanying claims and equivalents thereof, and any alterations equivalent to the accompanying claims are within the scope of the present invention.

That is, the described embodiments are completely for a description, and it should not be interpreted in any way to limit the scope of the present invention. The scope of the present invention is only defined by within the scope of the accompanying claims, and all various substitutions, changes in form and alterations derived from the meaning, scope and equivalents are within the scope of the present invention.

What is claimed is:

1. A tube shaped carbon structure comprising:
C₇₀; and
mesitylene,
wherein C₇₀ molecules have hexagonal structures, the mesitylene is bonded between the C₇₀ molecules and the hexagonal structures are regularly combined to form a tube shape.

2. The tube shaped carbon structure of claim 1, wherein a mole ratio of the C₇₀ to the mesitylene is 1:0.7.

3. The tube shaped carbon structure of claim 1, wherein the tube shaped carbon structure has an average diameter of 0.2 μm to 3.0 μm and an average length of 2 μm to 80 μm.

4. The tube shaped carbon structure of claim 1, wherein the tube shaped carbon structure has a single crystal structure.

5. A method of manufacturing a tube shaped carbon structure, the method comprising:
adding a carbon powder into a mesitylene solution and irradiating an ultrasonic wave to dissolve the carbon powder; and
adding an isopropyl alcohol solution into the mesitylene solution, in which the carbon powder is dissolved, by a volume ratio of 1:15 to 1:64, irradiating the ultrasonic wave and maintaining a stationary state.

6. The method of claim 5, wherein the carbon is C₇₀.

7. The method of claim 5, wherein a carbon concentration in the mesitylene solution is 0.1 mg/mL to 0.3 mg/mL.

8. The method of claim 5, wherein a mole ratio of the C₇₀ to the mesitylene is 1:0.7 in the tube shaped carbon structure.
